(19) United States
(12) Patent Application Publication
(10) Pub. No.: US 2003/0018365 A1
(43) Pub. Date: Jan. 23, 2003

(54) Method and apparatus for the treatment of urinary tract dysfunction

(76) Inventor: Gerald E. Loeb, South Pasadena, CA (US)

Correspondence Address: Attn: Marc E. Brown, McDERMOTT, WILL & EMERY, 34th Floor, 2049 Century Park East, Los Angeles, CA, 90067, US

(21) Appl. No.: 10/200,273

(22) Filed: Jul. 22, 2002

Related U.S. Application Data
(60) Provisional application No. 60/306,992, filed on Jul. 20, 2001.
(60) Provisional application No. 60/307,725, filed on Jul. 25, 2001.

(51) Int. Cl. A61N 1/34

(52) U.S. Cl. 607/40

(57) ABSTRACT

Electrical stimulation of specific sensory nerves to control the filling and/or emptying of the urinary bladder. A wireless, injectable microstimulator is implanted into the soft tissues through which the sensory nerves pass, but where they are not normally accessible by conventional open surgical implantation of conventional electrical stimulators or electrodes with leads. In males, the dorsal penile nerves 6 are stimulated by a microstimulator injected into the dorsal quadrant of the penis. The activity induced in these nerves cause the spinal cord to generate reflex responses that result in relaxation of the detrusor muscle, increasing bladder capacity and preventing incontinence as a result of inappropriate bladder contractions. The sensory nerves, such as urethral afferents 4, supplying the urethra are stimulated by a microstimulator implanted into the corpus of the penis, adjacent to the urethra. The activity induced in the urethral afferents 4 cause the spinal cord to generate reflex responses that result in contractions of the detrusor muscle and relaxation of the sphincter 5, emptying the bladder.

### CROSS-REFERENCE To RELATED APPLICATIONS

This application claims priority to and incorporates by reference two prior United States Provisional Applications entitled, Method and Apparatus for the Treatment of Urinary Tract Dysfunction: Serial No. 60/306,992, filed Jul. 20, 2001, and Serial No. 60/307,725, filed Jul. 25, 2001.

### BACKGROUND OF THE INVENTION

1. Field of the Invention

The invention relates to methods and associated apparatus which are useful for the treatment of urinary tract dysfunction. More particularly, the invention is directed to the use of an apparatus to control the filling and/or emptying of the bladder.

2. Background and State of the Art

Various dysfunctions of the urinary tract and its associated muscles and nerves result in the common clinical problem of urinary incontinence. Such dysfunctions may arise spontaneously in otherwise healthy individuals, but they are particularly common after various forms of damage to the spinal cord. The resulting incontinence interferes with the social life and health care of the patient. The neural mechanisms responsible for these dysfunctions are not fully understood.

The bladder acts as a storage reservoir for urine generated by the kidney. The bladder walls contain a muscle called the detrusor, which contracts to generate pressure and expel urine. The bladder wall also contains stretch receptors, which send signals about the distension of the bladder to the spinal cord. The interval of time between episodes of urination depends on the available volume of the reservoir. In normal adults, the capacity of the bladder is at least 500-700 cc. As the bladder starts to approach this capacity, the spinal cord reacts to the signals from the stretch receptors by activating the detrusor muscle. A person with an intact nervous system will be aware of both the distension and the pressure produced by the muscle contractions. If it is inconvenient to urinate, the person can voluntarily contract the sphincter muscle to prevent urination until it is convenient to do so. Urination is permitted to occur by relaxing the sphincter.

Patients with damage to the spinal cord suffer from various dysfunctions of the processes described above. Commonly, these patients suffer from spasticity of the detrusor muscle as a result of hyperactive reflex circuits in the spinal cord. Very small volumes of urine in the bladder trigger inappropriate bladder contractions. Often these patients are unable to sense or respond voluntarily to these contractions to prevent undesirable flow of urine. In other patients, bladder contractions result in an excessive reflexive response of the sphincter muscles, preventing urination even when desired. Still other patients fail to produce active bladder contractions even when the bladder is full; urine leaks out by overflow and the bladder never empties fully. Many patients suffer from a combination of these dysfunctions.

Many strategies have been proposed to use electrical stimulation to alter and correct dysfunction of the neuromuscular components of the urinary tract, some of which are now used in clinical practice. Conventional technologies for stimulating nerves include transcutaneous magnetic fields (induction of eddy currents in tissues by intense, pulsed magnetic fields created in externally affixed induction coils), transcutaneous electrical currents (applied via electrodes affixed to the skin or inserted into the vagina or rectum), percutaneous electrical currents (via wires injected through the skin that can be connected to external electronic stimulators) and fully implanted stimulators (pacemaker-like devices with leads routed subcutaneously to stimulating electrodes surgically affixed to the target structure).

Because the nerves that control bladder function are located deep in the pelvis, transcutaneous magnetic and electrical stimulation are often unacceptable because of the many other excitable nerves located superficial and adjacent to the target nerves. Percutaneous wires are usually unacceptable for chronic use, particularly in the perineal region of the body. Research to date has focused on surgical implantation of stimulating electrodes in, on or near main nerve trunks such as the pudendal nerve or the spinal cord itself. This requires the surgical routing of electrical leads from the electrodes to implanted electrical stimulators similar to cardiac pacemakers. Such surgical intervention is often feasible only for relatively large nerves that happen to run in places where they can be approached without endangering adjacent delicate or vital structures. Sites suitable for such intervention include the pudendal nerve as it passes the ischium, the spinal roots as they pass through the sacral foramena, and the spinal cord within the dural sheath. One general disadvantage of all of these sites is that they contain a mixture of neurons subserving various sensory and motor functions. This often makes it difficult to achieve the desired effects without producing undesirable side effects from inadvertent stimulation of inappropriate neurons. Another common disadvantage is that they generally require surgical intervention to implant the required devices, which entails high costs and risks of morbidity.

A new class of injectable microstimulators, the BION™ microstimulator, makes it possible to create accurately localized and precisely graded electrical fields within virtually any body structure. Each microstimulator includes electrical stimulation circuitry and electrodes configured in a form that is suitable for injection through a hypodermic needle. There are no attached leads to receive power or commands or to route stimulation pulses to distant electrodes. Microstimulators receive power by inductive coupling to an externally applied radio frequency (“RF”) magnetic field. They receive digital command signals by detecting and decoding modulations of the RF carrier. The electronic circuitry in the microstimulator may use the power and data immediately to generate the required electrical stimulation currents in the adjacent tissue by passing current through the integral electrodes, or it may store power and data by various conventional means to enable the generation of output pulses when the RF field is not present. The packaging and materials of the microstimulator are selected and designed to protect its electronic circuitry from the body fluids and to avoid damage to the electrodes and the surrounding tissues from the presence and operation of the microstimulator in those tissues. The use of microstimulators to induce, maintain and control micturition while being implanted in the corpus of the penis has not adequately been addressed by the prior art.

### SUMMARY OF THE INVENTION

The invention includes one or more microstimulators injected into soft tissues of the pelvis to activate reflex mechanisms in the spinal cord that modulate the state of the muscles that control pressure and flow in the bladder and urethra. In one embodiment, one microstimulator is located in the vicinity of the urethra where it can excite sensory fibers, such as urethral afferents, whose reflex actions tend to initiate or promote contraction of the bladder and relaxation of the sphincter. In another embodiment, a second microstimulator is located in the vicinity of the dorsal penile or clitoral nerve, whose activation tends to elicit reflexes that inhibit bladder contractions. Advantageously, in males both sites can be served by microstimulators positioned near the base of the penis and aligned axially with the long axis of the penis and with each other, permitting both implants to be powered and controlled by a small circumferential coil suitable for placement around the base of the penis. In one embodiment, this coil is larger in size to allow penetration of the magnetic field required to power a microstimulator which is more deeply implanted in the perineum.

### BRIEF DESCRIPTION OF THE DRAWING

FIG. 1. illustrates the components of one embodiment of the invention.

### DETAILED DESCRIPTION OF ILLUSTRATIVE EMBODIMENTS

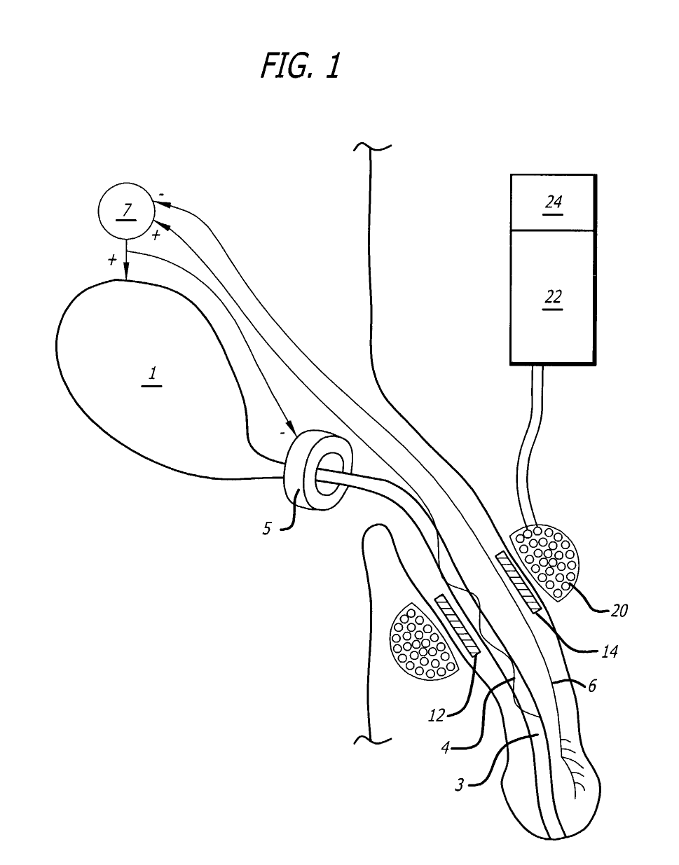

Referring to FIG. 1, the components of the lower urinary tract include the bladder 1, the urethra 3 and the sphincter 5. These structures are innervated by several different nerves subserving a wide range of sensory and motor functions that are interconnected in the spinal cord by many different spinal circuits 7. Only the most pertinent components are described herein and the details of their functions have been greatly simplified. Urethral afferents 4 convey sensory information from the urethra 3 to the spinal circuits 7 whose reflex outputs tend to excite contraction of the bladder 1 and reduce activity in the sphincter 5. Once they leave the vicinity of the urethra 3, the anatomical course of the urethral afferents 4 is not known and may not be surgically accessible in isolation. However, in the present invention, electrical stimulation is applied in the vicinity of the urethra 3 itself by a first microstimulator 12 implanted immediately adjacent to the urethra 3. The external surface of the penis is innervated by the two dorsal penile nerves 6 that run in parallel along the dorsal surface of the penis. These are electrically stimulated by a second microstimulator 14 implanted between or adjacent to the dorsal penile nerves 6, which results in an inhibition of spinal circuits 7 and a consequent prevention or reduction of bladder contraction. The microstimulator's 12 and 14 receive power and command signals through a receiving antenna inside the microstimulator which is typically a coil 20. The command signal is transmitted by the inductive coupling of a modulated alternating magnetic field created by a transmitting antenna, which in one embodiment is a coil. Each microstimulator has a different address, so it responds only to the command signals intended for it. The electrical signals required to generate this magnetic field are produced by a control unit 22, whose state depends on input from the patient received via user-activated control switch 24. Various specific methods and electronic circuits required to achieve the required functionality of the external and implanted elements (12, 14, 20, 22 and 24) are well-known and well-described in the prior art. For example, see U.S. Pat. Nos. 5,324,316 and 5,405,367 and Exhibit 1: Gerald E. Loeb, et al., “BION™ System for Distributed Neural Prosthetic Interfaces,” Medical Engineering and Physics 23: 9-18 (2001); Younghee Lee et al., “Detrusor and Blood Pressure Responses on Dorsal Penile Nerve Stimulation During Hyper-Reflexic Contraction of Bladder in Patients with Cervical Cord Injury,” Proceedings of the 6^{th} Annual Conference of the International Functional Electrical Stimulation Society Oral Session II: Neural Prostheses II: Sensory & Organ Systems; Kenneth J. Gustafson & Warren M. Grill, “Bladder Contractions Evoked by Electrical Stimulation of Pudendel Afferents in the Cat,” Proceedings of the 6^{th} Annual Conference of the International Functional Electrical Stimulation Society Oral Session II: Neural Prostheses II: Sensory & Organ Systems; John K. Chapin & Karen A. Moxin, “Neural Prostheses for Restoration of Sensory and Motor Function,” Gerald E. Loeb and Frances J. R. Richmond, BION™ “Implants for Therapeutic and Functional Electrical Stimulation,” incorporated by reference herein.

Still referring to FIG. 1, the invention relates to applying continuous or intermittent stimulation in a regular pattern to the dorsal penile nerves 6 via the microstimulator 14. One such pattern is a train of pulses at 20 pps for 30 seconds followed by a 30-second pause. The pause preferably prevents the spinal circuits 7 from habituating to the stimulation but is not so long as to allow the reflex inhibition of the bladder to wear off. The amplitude of the stimulation pulses can be set initially by the prescribing therapist by observing the reflexive contraction of the pelvic floor muscles that tends to be elicited by activation of the dorsal penile nerves 6. When the patient is ready to urinate, a user-activated control switch 24 permits him to change the state of the control unit 22 so that stimulation of the dorsal penile nerves 6 is discontinued and stimulation of the urethral afferents 4 begins. A suitable pattern is a train of pulses at 2 pps until the bladder is empty and the flow of urine ceases. The amplitude of the stimulation pulses is set initially by the prescribing therapist in a urodynamic examination by determining the level that results in reflexive contraction of the bladder, as determined by measuring increases in bladder pressure when the bladder is full.

Another embodiment provides for intermittent stimulation of the urethral afferents 4 alone. In this embodiment, only the first microstimulator 12 is implanted adjacent to the urethra 3 to stimulate the urethral afferents 4 alone. This location is suitable for intermittent inductive powering at the time of urination, without requiring a coil 20 to be worn at other times. Stimulation of this type is suitable for males and females, particularly if undesirable sphincter 5 contractions need to be avoided or minimized, such as is the case in females.

An additional function of the invention is the ability for a patient to switch between an excitatory (urethral) and inhibitory (dorsal penile) reflex effects on bladder contractions using a user-activated control switch 24 on a daily basis in order to inhibit undesired micturition and initiate and complete micturition desired micrturition. Additionally, the switch may be used to activate a change in stimulation patterns needed by patients that have a mix of dysfunctions which change over time in response to progression of their underlying neurological problems and plastic changes in the genitourinary tract, resulting from chronic use of the treatment disclosed herein.

Advantageously, the control unit 22 includes a storage device whereby one or more programs of stimulation pulses that have been devised by the therapist are retained electronically and generated as required by the patient. This invention may be combined with other technology, such as a sensor that can detect or distinguish bladder fullness in order to alert the user to the need to empty the bladder. In another embodiment, this invention is practiced with microstimulator implants that have storage for power and stimulation parameters so that they can generate stimulation pulses even when coil 20 is not physically present. In that case, the coil 20 and control unit can be used intermittently to provide power to recharge power storage such as rechargeable lithium ion cells and to transmit data regarding the required stimulation parameters.

Furthermore the present invention utilizes the advantageous geometry of the penis, which affords a small, energy efficient and easily worn transmission coil 20 which is of a circumference that allows the coil 20 to easily wrap around the penis. In one embodiment, a single coil 20 controls both the excitatory and inhibitory stimulation sites adjacent to each other where the peripheral nerves are parallel but still anatomically separate. Microstimulators 12 and 14 that receive all of their power from the coil 20 can be substantially smaller and hence easier to inject into delicate structures such as the penis because they do not require internal power storage cells.

However, although the invention thus far described has focused predominately on male patients, correspondences to the female anatomical structure are well-known. The dorsal clitoral nerve of the female serves a similar genital sensory function and is known to have similar inhibitory reflexes on bladder contraction as the dorsal penile nerve 6 in males. The microstimulator implants are small enough to be injected into the subcutaneous tissues lateral and proximal to the clitoris. The female urethra is shorter and for most of its length is surrounded by the sphincter muscles. A microstimulator implant in or near the female sphincter tends to stimulate both the desired urethral afferents in the female and the motor neurons that produce sphincter contraction; the latter counteracts the desired outcome of unobstructed micturition. Fortunately, the spinal micturition reflex elicited by the urethral afferents responds best to very low frequencies of stimulation (1-2 pps), whereas such stimulation would produce only brief, weak twitches of the sphincter that would not interfere significantly with urine flow.

In another embodiment, it may be more effective if the stimulation is directed toward urethral afferents arising more proximally from the region of the urethra within the sphincter 5 and between the sphincter 5 and the bladder 1. In that case, the microstimulator 12 should be implanted in this site, which lies near the prostate gland in the male. For this purpose, coil 20 would probably need to be somewhat larger in diameter than depicted in FIG. 1 in order to generate sufficient magnetic field strength to power microstimulator 12 in these deeper tissues of the pelvis.

The descriptions of exemplary and anticipated embodiments of the invention have been presented for the purpose of illustration and description. They are not intended to be exhaustive or to limit the invention to the precise forms disclosed. For example, the invention may include a multiplicity of injectable microstimulators which induce and/or maintain micturition by stimulating urethral afferents or both induce and inhibit micturition by stimulating urethral afferents and the dorsal penal or dorsal clitoral nerves. In short, the invention is limited solely by the claims that follow.

1. An apparatus for exciting urethral afferents to induce micturition comprising:
a) a first injectable microstimulator comprising:
1) a chamber separating a plurality of exposed electrodes for delivering controllable electrical current in the area of the urethral afferents;
2) an electronic circuit within said chamber in communication with said electrodes for generating the controllable electrical current in response to control signals;
3) a receiving antenna within said chamber in communication with said electronic circuit for receiving control signals;
b) a control unit for generating the control signals; and
c) a transmitting antenna in communication with said control unit for transmitting the control signals to said receiving antenna.

2. The apparatus for exciting urethral afferents to induce micturition as in claim 1 wherein said first injectable microstimulator is adapted for implantation in the corpus of the penis.

3. The apparatus for exciting urethral afferents to induce micturition as in claim 1 wherein said injectable microstimulator is adapted for implantation in the perineum.

4. The apparatus for exciting urethral afferents to induce micturition as in claim 1 further including a second injectable microstimulator that is injectable in an area in a human body which inhibits micturition.

5. The apparatus for exciting urethral afferents to induce micturition as in claim 1 further including a second injectable microstimulator wherein said first injectable microstimulator and said second injectable microstimulator are adapted for implantation into the base of a penis, wherein said first injectable microstimulator is adapted for axial alignment with said second injectable microstimulator within the penis and with the long axis of the penis.

6. The apparatus for exciting urethral afferents to induce micturition as in claim 4 wherein said second injectable microstimulator is adapted for stimulation of the dorsal penile nerve.

7. The apparatus for exciting urethral afferents to induce micturition as in claim 4 wherein said second injectable microstimulator is adapted for stimulation of the dorsal clitoral nerve.

8. The apparatus for exciting urethral afferents to induce micturition as in claim 1 wherein said transmitting antenna is a coil of a circumference suitable for positioning around the base of the penis for controlling said first injectable microstimulator and a second injectable microstimulator.

9. The apparatus for exciting urethral afferents to induce micturition as in claim 1 further comprising a user-activated control switch for initiating stimulation of the urethral afferents in order to induce or maintain micturition.

10. An apparatus for exciting urethral afferents to induce micturition comprising an injectable microstimulator having a plurality of exposed electrodes for delivering controllable electrical current in the area of the urethral afferents which cause the urethral afferents to induce micturition.

11. An apparatus for exciting urethral afferents to induce micturition comprising:
a) a first microstimulator implanted in proximity to urethral afferents having a chamber separating a plurality of exposed electrodes for delivering controllable electrical current in the area of the urethral afferents in response to control signals and a receiving antenna within said chamber for receiving control signals;
b) a control unit for generating the control signals; and
c) a transmitting antenna in communication with said control unit for transmitting the control signals to said receiving antenna.

12. The apparatus for exciting urethral afferents to induce micturition as in claim 11 wherein the first microstimulator is implanted near the base of a penis and is axially aligned with a second microstimulator and the long axis of the penis, wherein the second microstimulator is implanted in an area of the human body that inhibits micturition.

13. The apparatus for exciting urethral afferents to induce micturition as in claim 11 wherein said transmitting antenna is a coil 20, wherein said coil 20 is of a circumference suitable for positioning around the base of the penis and adapted for controlling said first microstimulator and a second microstimulator.

14. The apparatus for exciting urethral afferents to induce micturition as in claim 11 further comprising a user-activated control switch for initiating stimulation of the urethral afferents in order to induce or maintain micturition.

15. A method for exciting urethral afferents to induce micturition comprising:
a) generating electrical signals by a control unit;
b) delivering the signals to a transmitting antenna;
c) receiving signals by a receiving antenna within a first microstimulator in the area of urethral afferents;
d) generating electrical signals in the first microstimulator in the area of urethral afferents; and
e) inducing micturition.

16. The method for exciting urethral afferents to induce micturition as in claim 15 further comprising delivering electrical signals to urethral afferents through a multiplicity of microstimulators.

17. The method for exciting urethral afferents to induce micturition as in claim 15 further comprising inhibiting micturition with a second microstimulator that receives signals from the control unit and generates signals for inhibiting micturition.

18. The method for exciting urethral afferents to induce micturition as in claim 15 further comprising initiating stimulation of the urethral afferents in order to induce or maintain micturition.

19. The method for exciting urethral afferents to induce micturition as in claim 15 wherein the first microstimulator is implanted in the corpus of the penis.

20. A method for creating an electronic interface to urethral afferents to induce micturition comprising injecting a first microstimulator into a human recipient in the area of urethral afferents.

21. The method for creating an electronic interface to urethral afferents to control micturition as in claim 20 further comprising injecting a second microstimulator for delivering electrical signals which inhibit micturition.

22. The method for creating an electronic interface to urethral afferents to control micturition as in claim 20 wherein said injecting is through a hypodermic needle.

23. A method for creating an electronic interface to control micturition comprising injecting an injectable microstimulator into the corpus of a penis.
